(19) United States
(12) Patent Application Publication
(10) Pub. No.: US 2012/0095440 A1
(43) Pub. Date: Apr. 19, 2012

(54) INJECTION PORT SYSTEM FOR INTRA-MARROW INJECTION/INFUSION

(76) Inventor: Abul Bashar Mohammad Anwarul Islam, Amherst, NY (US)

(21) Appl. No.: 13/270,488

(22) Filed: Oct. 11, 2011

(30) Foreign Application Priority Data
Oct. 13, 2010 (GB) 10 17247.6

(51) Int. Cl. A61M 5/00

(52) U.S. Cl. 604/506; 604/175

(57) ABSTRACT

An injection port system for placement in or on an individual includes a first port (55) defining a first injection chamber (58) with a septum (59), and a tubular securing device (38) for securing said first injection chamber to bone (52) of the individual. A needle inserted through the septum (59) enables material to be injected into or removed from the bone marrow (60), for example injecting chemotherapeutic agents, or stem cells, or withdrawing bone marrow for analysis. The injection port system (50) also comprises a second port (62) defining a second injection chamber (65) with a septum (66), the first port (55) being mechanically connected to the second port (62). The second port (62) is linked by a catheter (68) to a blood vessel, so that therapeutic fluids can be directly supplied to the blood system, and blood samples can be taken for monitoring.

The current invention relates to an injection port system for intra bone marrow injection/infusion of materials for the treatment of individuals.

It is known to introduce drugs into individuals by way of injection ports into blood vessels and these can be used not only to administer drugs to an individual but also to take samples from those individuals. Such ports are implanted under the skin of an individual and are sutured into position on soft body tissue. The port is formed of a hollow chamber sealed by a soft top called a septum which lies under the skin and in turn, the port is connected to a catheter which extends from the port under the skin into a blood vessel. The port may be located in one part of the body, such as near the sternum or clavicle where it is readily accessible by healthcare professionals and yet is comfortable for the patient and which does not restrict their daily activities

Such a device may be situated so that materials can be injected into major blood vessels of the individual. One application of these ports is the introduction of chemotherapy agents for the treatment of conditions such as leukaemia and other malignancies. Drugs or chemotherapeutic agents, when administered intravenously through blood vessels, are diluted when they mix with the blood and at some point they may be subjected to alteration and deactivation by the liver and other organs before the agents reach the bone marrow that is contained within the bones. The bone marrow has different types of cells e.g. pluripotential stem cells, that give rise to red cells, white cells and platelets for blood which travels round the body. The marrow may also contain abnormal cells that are not normally present, such as cancer cells (metastatic or non-metastatic). In addition, once in circulation, the drugs may also bind specifically or non-specifically to proteins or other agent and therefore become less effective or even totally inactivated, which is disadvantageous because it reduces the effectiveness of treatment.

It has therefore been proposed to provide devices whereby drugs or fluids can be directly fed into bone marrow. For example U.S. Pat. No. 4,772,261 (Von Hoff et al) described an intramedullary catheter comprising a tubular conduit which may have an external thread for attachment to a bone, the conduit having an enlarged head with a self-sealing membrane. The device would be installed in a hole drilled through a bone into communication with the marrow cavity, so that the open end of the device is within the bone marrow while the head of the device is at the outside of the bone, and would then be covered over by the patient's skin. A similar device is described in U.S. Pat. No. 6,228,088 (Miller et al).

According to a first aspect of the present invention there is provided an injection port system for placement in or on an individual, said injection port system comprising a first port defining a first injection chamber with a septum, and a tubular securing device for securing said first injection chamber to bone of the individual, said septum forming a puncture area for a needle to inject/infuse material by way of the first injection chamber to said tubular securing device which, in use, is inserted through the bone and into the bone marrow cavity, said tubular securing device defining at least one aperture so that material can be injected into or removed from the bone marrow, wherein the injection port system also comprises a second port defining a second injection chamber with a septum, the first port being mechanically connected to the second port, and means to provide fluid communication between the second injection chamber and a blood vessel.

This injection port system including two separate injection chambers, each with a septum, simplifies medical treatment of a patient where provision of drugs or fluids into both bone marrow and directly into the blood system is required. At the same time it ensures that the second port is held in position within the patient's body, by virtue of the mechanical connection between the first port and the second port, and the tubular securing device that holds the first port to the bone.

For example, the injection port system may be secured to the bone forming the iliac crest of the individual, for example the anterior or the posterior iliac crest. Alternatively the injection port system may be attached to the sternum.

In a preferred arrangement, the septum is elevated to provide a readily identifiable area, which being raised under the skin is visible or palpable by healthcare professionals so they have a clear and identifiable target for any needle being injected into the port. Each injection port may be provided with raised protrusions on its outer surface, so the healthcare professionals can more readily locate the ports, and distinguish between the two ports. However, it is also envisaged that the septum may be flat so it lies unobtrusively beneath the skin. Preferably the septum is silicone or latex type material which allows for repeated puncture.

The tubular securing device can be inserted into the bone of an individual. It may comprise a screw that can be screwed through the bone so the tip of the screw projects into the bone marrow cavity. The screw may be an orthopaedic screw which can remain in the individual's body for an indefinite period of time and the screw has helical threads that preferably are chamfered to enable the screw to cut through the bone as it is inserted. The helical threads also provide securing means for holding the screw in position in the bone. By having a device that fixes directly into bone, this provides a very secure location for a port, which can remain in position for many months or even indefinitely if the individual has a chronic condition.

Alternatively the tubular securing device may be of substantially uniform diameter, with a tapered point. The portion of the device of uniform diameter may define surface recesses or grooves, which may be circumferential or helical. Friction between such surface features and the surrounding bone reduces the risk of the device being unintentionally removed from the bone; and after implantation the bone will grow into the recesses or grooves, so the tubular securing device is firmly anchored to the bone. Rotation of the tubular securing device may not be convenient where there are two adjacent injection ports, and such devices of substantially uniform diameter do not have to be rotated during insertion, although during insertion it may be advantageous to twist the tubular securing device to and fro through a small angle.

The tubular securing device has a lumen passing down its length, which may be sufficiently large to receive a needle that has been inserted through the septum, passing through the injection chamber and into the securing device. The needle may locate just at the start of the lumen or, in some instances, the needle can pass at least part way down the lumen of the securing device. The lumen passes through the securing device and there are one or more apertures in the wall of the securing device forming a passage from the lumen to the outside of the securing device which allow for release of materials, or if materials are being withdrawn from an individual the apertures allow material to pass into the securing device which are then withdrawn through a needle in the port system. The apertures may be along at least part of the length of the securing device or there may be just one aperture at the tip of the securing device, or a combination of both. It is also envisaged that rather than a needle engaging with the tubular securing device, the needle may inject material directly into the injection chamber so the material flows through the lumen of the securing device and out into the bone marrow.

The fluid communication between the second injection chamber and a blood vessel is preferably provided by a flexible catheter.

In one arrangement the tubular securing device has a head which has recesses. The recesses can mate with protrusions on a driving device for inserting the securing device into bone. The securing device can be screwed into the bone by twisting the driving device which in turn twists the securing device. As an alternative the tubular securing device may define pegs or detents, which mate with recesses on a driving device.

According to another aspect of the invention there is provided a method of treatment of a patient, the method comprising installing an injection port device into the patient, the injection port device comprising a first port defining a first injection chamber with a septum, and a tubular securing device for securing said first injection chamber to bone of the individual, said septum forming a puncture area for a needle to inject material by way of the first injection chamber to said tubular securing device, the installing step comprising inserting the tubular securing device through a bone in the patient and into a bone marrow cavity, said tubular securing device defining at least one aperture so that material can be injected into or removed from the bone marrow, wherein the method then comprises injecting stem cells into the bone marrow.

This method has the advantage that the stem cells are directly supplied/implanted to the bone marrow.

The method of treatment specified above may use the injection port system described earlier, providing both a first injection chamber communicating with the bone marrow and a second injection chamber communicating with a blood vessel. In this case the method may also comprise supplying drugs or other materials directly into the blood system simultaneously with injecting human stem cells into the bone marrow. For example the method may comprise transfusion of blood or blood products, such as plasma or blood platelets, or infusion of fluids or immunoglobulins into a blood vessel, for example the jugular vein or subclavian vein.

The method of treatment of the invention improves the ability to deliver specific therapy directly into the marrow cavity and with minimum side effects. It may therefore be applicable to diseases such as leukaemia, lymphoma and other malignant conditions of the marrow such as multiple myeloma and myelodysplastic syndromes. This method of treatment can also be used in malignant conditions which particularly metastasize in the bone marrow such carcinomas of the lung, breast or prostrate. Also, by providing a securely implanted device into the marrow, this allows individuals to be effectively treated on an out-patient basis, which is very cost effective. This would be a major improvement from the patient's point of view, minimising the need for hospitalization, thereby improving the patient's quality of life. It is to be noted that the invention is applicable not only to the treatment of humans but also to the treatment of animals.

Preferably the method of treatment includes installing the injection port device in the sternum. A benefit of installing the device in the sternum is that it is unlikely to be exposed to abrasion from clothes. Furthermore the sternum is readily accessible, and the bone is not unduly hard.

The present invention, in a further aspect, provides an injection port device suitable for this method of treatment.

The invention will now be further and more particularly described by way of example only and with reference to the accompanying drawings in which:

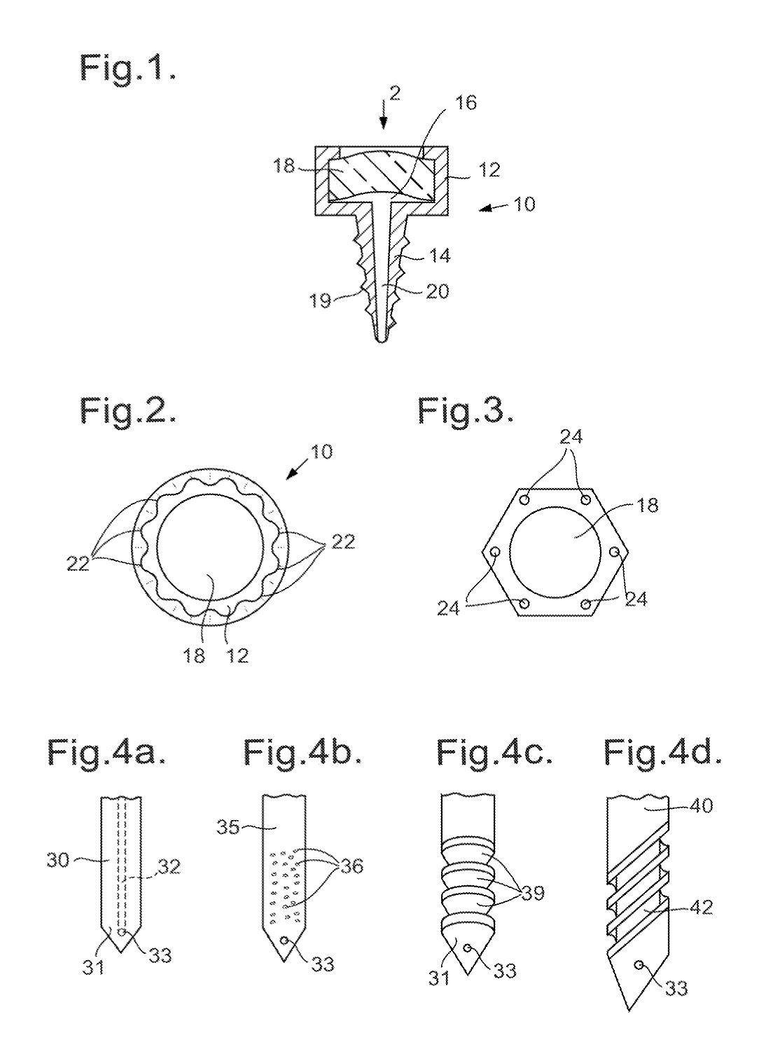

FIG. 1 shows a sectional view of an injection port device for injecting stem cells into the marrow of a patient's sternum;

FIG. 2 shows a view from above, in the direction of arrow 2, of the injection port device of FIG. 1;

FIG. 3 shows a view from above of a modification to the injection port device of FIG. 1;

FIGS. 4a to 4d show modifications to part of the injection port device of FIG. 1;

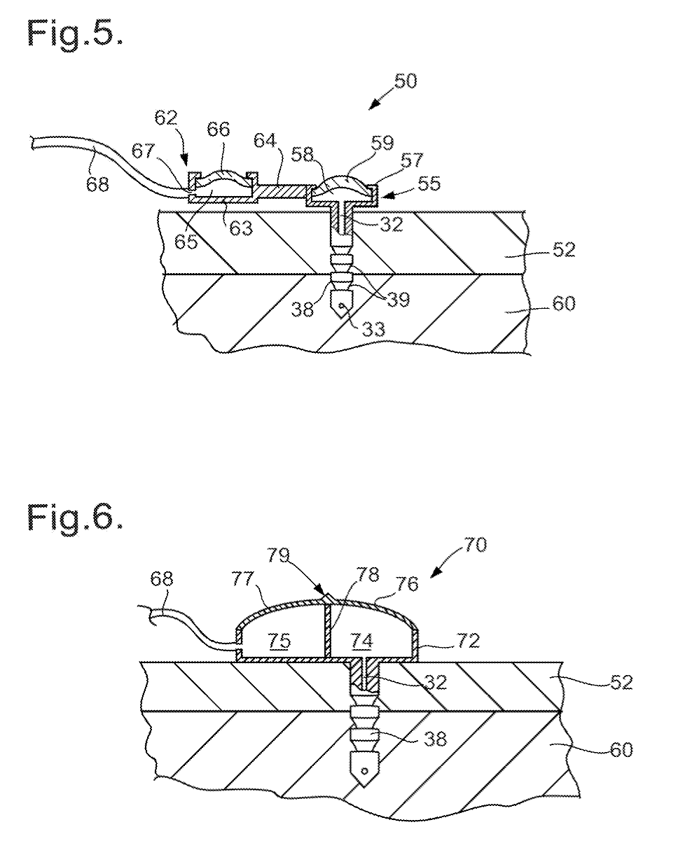

FIG. 5 shows a sectional view of an injection port system of the invention; and

FIG. 6 shows a sectional view of an alternative injection port system of the invention.

Referring now to FIGS. 1 and 2, an injection port device 10 consists of a head portion 12 from the underside (as shown) of which projects a tapered portion 14. The head portion 12 is hollow, and defines an injection chamber 16 enclosed by a flexible septum 18. The tapered portion 14 defines a helical thread 19 on its outer surface, and defines a lumen 20 along its length. As shown in FIG. 2, the outer surface of the head portion 12 is provided with several ridges 22.

The injection port device 10 is intended for use in a patient's sternum. In use, after cleaning the skin covering the sternum, a surgeon would make an incision, and separate the skin on either side of the incision so as to expose the outer surface of the sternum. A trocar may be used to form a hole through the sternum into the marrow cavity. The injection port device 10 would then be screwed into that hole until the underside of the head portion 12 was resting against the outer surface of the sternum. The length of the tapered portion 14 is such that the open end of the lumen 20 communicates with the marrow cavity of the sternum. To complete the installation, the two halves of the incision would then be brought back together over the top of the injection port device 10. The edges of the wound would then be stitched together, and a dressing applied. Once the wound is healed, the injection port 10 is ready for use. Once installed, the helical thread 19 ensures that the injection port 10 remains securely fixed to the sternum. The septum 18 is of a material that can be repeatedly punctured by a needle (when injecting or withdrawing fluids from the chamber 16), and which reseals when the needle is withdrawn.

The outer surface of the head portion 12 may have a different shape, although preferably in every case the outer surface provides a grip to help the surgeon twist the injection port device 10. For example, as shown in FIG. 3, the outer surface of the head portion 12 may instead be hexagonal. Furthermore the top surface, which after installation is immediately under the skin, may be provided with bumps or protrusions 24 (as shown in FIG. 3) to make it easier subsequently to locate the injection port device 10, and in particular the centre of the septum 18, by palpation

The injection port device 10 is particularly intended for the direct injection of haemopoietic stem cells into bone marrow, although it may also be used for chemotherapy or other fluids. A known treatment for conditions that include leukaemia, multiple myeloma and non-Hodgkin's lymphoma is referred to as bone marrow transplantation. This entails collecting haemopoietic stem cells from cord blood, or from peripheral blood (by collecting a small number of circulating CD 34 positive stem cells), or from bone marrow from donors, and injecting them into patients after ablative therapy. However, where such stem cells are injected into the bloodstream they may be trapped in tissues or organs other than bone marrow, and so will have no therapeutic effect. Furthermore there is a risk that the injected stem cells may be destroyed by circulating macrophages. There is also a risk that in circulation some of the stem cells may come into contact with chemicals that are detrimental to them. The injection of haemopoietic stem cells or harvested bone marrow cells directly into the marrow cavity by using a port such as the injection port device 10 avoids all the risks and drawbacks that arise when the injection is into the bloodstream.

An injection port device of the invention may be affixed to the bone by means other than a helical screw thread. This may be particularly advantageous where repeated rotation of the injection port device during installation is to be avoided.

For example, referring now to FIG. 4, this shows different designs that may be used in place of the tapered portion 14 with the screw thread 19 of FIG. 1, which is installed by repeated rotation (as a conventional screw). In each example of FIG. 4 the design would extend from a head portion equivalent to the head portion 12 of FIG. 1; and in each case there is a shaft with uniform outer diameter, and a tapered tip.

The embodiment of FIG. 4a shows a side view of a shaft 30 of uniform outer diameter, with a conical pointed tip 31. A longitudinal lumen 32 communicates with an aperture 33 in the tip 31. FIG. 4b shows a side view of a shaft 35 which differs from the shaft 30 only in that the outer surface is provided with roughened portions 36. FIG. 4c shows a perspective view of a shaft 38 which defines circumferential grooves 39, the upper surface of each groove 39 being a frustro-conical surface parallel to that of the tip 31. FIG. 4d shows a side view of a shaft 40 of uniform outer diameter with a conical pointed tip 31. It differs from the shaft 30 in being provided with helical grooves 42 on part of the shaft 40.

The shafts 30, 35, 38 and 40 of FIG. 4 can be inserted into the bone simply by pushing on the head portion. The roughened portions 36 and the circumferential grooves 39 and 42 reduce the risk of the shaft being dislodged after installation, by increasing friction with the bone. After installation, bone cells will grow into these recesses or grooves, fixing the injection port device more firmly.

Referring now to FIG. 5 there is shown a cross-sectional view of an injection port system 50 installed in a patient's sternum 52. The system 50 comprises a first injection port 55 similar to that of FIG. 1, with a head portion 57 connected to a shaft portion 38 (as shown in FIG. 4c). The head portion 57 is hollow, defining a first injection chamber 58 enclosed by a septum 59. The first injection chamber 58 communicates with a lumen 32 or bore that extends to the aperture 33, which after installation is in the marrow 60 of the sternum 52. This first injection port 55 is similar to that of FIG. 1, but because it incorporates the shaft portion 38 it can be inserted without repeated turning, by pushing it through a hole formed using a trocar in the sternum 52. Because the sternal bone is not as hard as the bone in the anterior or posterior iliac crest, the use of a trocar to make a hole may not be necessary. The shaft portion 38 may be introduced by gentle pushing along with minor clockwise and anti-clockwise twisting of the head portion 57.

The injection port system 50 also comprises a second injection port 62 which comprises a body part 63 which is integral with the head portion 57, being linked to it by a bridge portion 64. The body part 63 defines a second injection chamber 65 which is covered by a septum 66. The second injection chamber 65 has an outlet port 67 which communicates via a flexible catheter 68 to a vein (not shown), for example the subclavian or jugular vein.

In a modification to the port system 50, the body part 63 and the head portion 57 are clipped or snapped together. After installing the head portion 57 in the patient, the body part 63 can then be clipped onto the head portion 57. This may make it easier to attach the head portion 57 to the bone. For example the bridge portion 64 may incorporate such a clip mechanism. The clip mechanism may be arranged such that the two components (body part 63 and head portion 57) can subsequently be separated, by releasing the clip. Alternatively the clip mechanism may be effectively irreversible, without any way of releasing the clip, so that after clipping the components together they cannot be separated. Such clip mechanisms, whether releasable or not releasable, are well-known to the skilled person, and for example may comprise a rod that slides into a tube, with resilient latches at the end of the rod which, on insertion, engage with corresponding recesses on the inside of a tube. This can only be released by releasing the latches from the recesses.

As with the septum 18, the septum 59 and the septum 66 are of a material through which a needle can be readily inserted to inject material into the respective injection chamber 58 or 65, and which seals again when the needle is withdrawn.

The injection port system 50 is installed in a similar way to that described above, with the shaft portion 38 fixing the injection port 50 onto the sternum 52, and the entire injection port system 50 then being covered over by the two sides of the incision, the wound then being stitched and dressed so the injection port system 50 is under the skin of the patient. Subsequently the first injection port 55 can be used for injecting stem cells into the bone marrow 60 of the sternum 52, or indeed for injection or infusion of chemotherapeutic agents directly into the bone marrow. The second injection port 62 may be used for transfusion of blood or blood products, such as plasma or platelets, or infusion of chemotherapeutic agents, or fluids, or immunoglobulins to boost immunity, these being provided into the blood vessel to which the catheter 68 is connected.

To ensure the septum 59 can be readily distinguished from the septum 66, the top surfaces of the head portion 57 and of the body portion 63 may be provided with projecting bumps (not shown), equivalent to the bumps 24 of FIG. 3. These may provide different geometrical shapes that can be readily distinguished by palpation. For example the head portion 57 might define six bumps arranged as a hexagon, while the body portion 63 might define three bumps arranged as an equilateral triangle. They may also be distinguished by the shapes of the septa, for example the septum 59 might be circular while the septum 66 might be rectangular with rounded corners. Such projecting bumps may alternatively or additionally be provided on the septa 59 and 66 rather than on the head portion 57 and the body portion 63.

Referring now to FIG. 6 there is shown a modified injection port system 70, in which a head portion 72 defines two separate injection chambers 74 and 75, each with a septum 76, 77 (equivalent to the septa 59 and 66). The injection chambers 74 and 75 are separated by a divider wall 78 which may be of plastic or metal, and which prevents any fluid communication between the chambers 74 and 75. One injection chamber 74 communicates with bone marrow through the lumen 32 of a shaft 38, while the other injection chamber 75 has an outlet port which communicates via a flexible catheter 68 to a vein. In this example the injection port system 70 includes a raised or palpable nipple-like bump 79 above the divider wall 78, so that the healthcare professional can determine where the boundary is between the two chambers 74 and 75, and so determine where to insert a needle. In this case the injection port system 70 must be installed with a consistent orientation. For example this may be with the injection chamber 75 and the flexible catheter 68 consistently being placed facing the patient's head (i.e. the cephalic side), and the injection chamber 74 on the side further from the head (i.e. non-cephalic, bottom, facing towards the leg). This system 70 may provide a somewhat smaller injection port system.

The system 70 after installation communicates with the marrow 60 of the sternum 52; and, because of the shape of the shaft portion 38, it can be inserted without repeated turning, by pushing it through a hole formed using a trocar in the sternum 52.

It will be appreciated that the injection port system 50 or 70, providing access both to bone marrow and to a vein has the benefit over the use of separate injection ports that it can be placed in one site with a single small surgical incision, for example over the sternum 52 and directly communicating with the sternum and with a blood vessel such as the subclavian vein. The access to the bone marrow 60 enables bone marrow sampling to be carried out repeatedly, as is required in monitoring illnesses such as leukaemia. It also enables such illnesses to be treated by direct infusion of chemotherapy drugs into the marrow, or direct injection of haematopoietic stem cells into the bone marrow 60. It also enables drugs to be introduced directly into a blood vessel, or for blood samples to be withdrawn.

Although the invention has particular applications in the treatment of humans, it also can be used in the treatment of animals.

Various changes and modifications to the embodiments shown in the drawings and described above may be within the scope of the invention as claimed. Furthermore where several embodiments are discussed, the invention is intended to cover combinations of those embodiments. Also it is therefore intended that all matter contained in the above description and shown in the accompanying drawings shall be interpreted as illustrative of the invention.

Thus the port in communication with the bone marrow may be used, for example, for: injecting/introducing chemotherapeutic/anticancer agents (such rituxan which is a monoclonal antibody); injecting/introducing haemopoietic stem cells for transplantation; and withdrawal of bone marrow for diagnosis and repeat sampling to monitor treatment as well as to see response to treatment/relapse etc. The port in communication with a blood vessel can be used, for example, for: injecting/introducing therapeutic fluids like blood, plasma, immunoglobulin, antibiotics etc.; withdrawal of blood to monitor haematologic (such as “complete blood count” (CBC)) and metabolic changes (such as “complete metabolic profile” (CMP)), blood cultures etc.; easy access to blood is very important in the treatment of cancer patients.

1. An injection port system for placement in or on an individual, said injection port system comprising a first port defining a first injection chamber with a septum, and a tubular securing device for securing said first injection chamber to bone of the individual, said septum forming a puncture area for a needle to inject/infuse material by way of the first injection chamber to said tubular securing device which, in use, is inserted through the bone and into the bone marrow cavity, said tubular securing device defining at least one aperture so that material can be injected into or removed from the bone marrow, wherein the injection port system also comprises a second port defining a second injection chamber with a septum, the first port being mechanically connected to the second port, and means to provide fluid communication between the second injection chamber and a blood vessel.

2. An injection port system as claimed in claim 1 wherein the first port and the second port can be distinguished by palpation.

3. An injection port system as claimed in claim 2 wherein the first port and the second port have different shapes in plan.

4. An injection port system as claimed in claim 2 wherein the first port and the second port are provided with protrusions arranged in arrays of different geometry.

5. An injection port system as claimed in claim 1 wherein the tubular securing device incorporates a tapered shaft with a screw thread so it can be inserted through the bone by repeated rotation in the same sense.

6. An injection port system as claimed in claim 1 wherein the tubular securing device incorporates a shaft of uniform diameter, so it can be inserted through the bone without repeated rotation in the same sense.

7. An injection port system as claimed in claim 1 wherein the fluid communication between the second injection chamber and a blood vessel is provided by a flexible catheter.

8. An injection port system as claimed in claim 1 wherein the first port and the second port are defined within a unitary head connected to the tubular securing device.

9. An injection port system as claimed in claim 1 wherein the second port is connected to the first port by a clip mechanism.

10. A method of treatment of a patient, the method comprising installing an injection port device into the patient, the injection port device comprising a first port defining a first injection chamber with a septum, and a tubular securing device for securing said first injection chamber to bone of the individual, said septum forming a puncture area for a needle to inject material by way of the first injection chamber to said tubular securing device, the installing step comprising inserting the tubular securing device through a bone in the patient and into a bone marrow cavity, said tubular securing device defining at least one aperture so that material can be injected into or removed from the bone marrow, wherein the method then comprises injecting stem cells into the bone marrow.

11. A method of treatment as claimed in claim 10 wherein the injection port device is attached to the patient's sternum.

12. A method of treatment as claimed in claim 10 wherein the injection port device is attached to the bone forming the iliac crest of the patient, either the anterior or the posterior iliac crest.

13. A method of treatment as claimed in claim 10 wherein the injection port device is an injection port system as claimed in claim 1, and the second injection chamber communicates with a blood vessel.

14. A method of treatment as claimed in claim 10 comprising simultaneously injecting stem cells into the bone marrow and supplying a liquid material directly to a blood vessel.

15. A method as claimed in claim 14 wherein the liquid material is selected from blood, blood plasma, blood platelets, and a liquid containing immunoglobulins.

16. A method as claimed in claim 13 wherein the blood vessel is the patient's jugular vein or subclavian vein.

17. An injection port device suitable for use in a method of treatment as claimed in claim 10.

18. An injection port device as claimed in claim 17 comprising a securing element for securing the device to a bone, the securing element defining a bore to communicate with the bone marrow, and the securing element being connected to a head element provided with angled faces or with ridges so it can be firmly gripped by medical staff during installation.
